(19) United States
(12) Plant Patent Application Publication
(10) Pub. No.: US 2002/0188996 P1
(43) Pub. Date: Dec. 12, 2002

(54) Petunia plant named 'Sunbelkuopi'

(76) Inventor: Yasuyuki Murakami, Gamou-gun (JP)

Correspondence Address: BURNS, DOANE, SWECKER & MATHIS, L.L.P., P.O. Box 1404, Alexandria, VA, 22313-1404, US

(21) Appl. No.: 09/878,239

(22) Filed: Jun. 12, 2001

(51) Int. Cl. A01H 5/00

(52) U.S. Cl. PLT/356

(57) ABSTRACT

‘SUNBELKUOPI’ is a decumbent plant, with small and vivid purplish pink flowers, which is suitable for potting, hanging pots and flower bedding. This plant has abundant branching and secondary branching is especially strong. Leaf shape is lanceolate to elliptic with some pubescence on the surface, having a small petiole. Leaf color is dark yellowish green (RHS C.C. No. 146A). The flowers are single and very small, 2.8cm in diameter. The color of flower is vivid purplish pink (RHS C.C. No.80C). The bottom color of inside of throat is bright greenish yellow (RHS C.C.No.2B) and the outside color of corolla tube is pale yellowish green (RHS C.C. No.150D). Flowering time is late. The plant has a high resistance to heat, rain, cold and diseases.

### BACKGROUND OF THE VARIETY

The present invention relates to a new and distinct variety of petunia plant obtained from crossing a wild type of petunia plant ‘CDS02’ native to Brazil, as a female parent, with a wild type of petunia plant variety ‘CDS01’, as a pollen parent.

The petunia is a very popular plant and is used for flower bedding and potting in the summer season. There are only a few varieties of the petunia plant which do not have an upright growth habit and which have a high resistance to rain, heat, cold, and diseases. The petunia previously filed, i.e., the “Revolution” series [(Revolution Purplepink(U.S. Pat. No, Plant 6,915), Revolution Brilliantpink (U.S. Pat. No, Plant 6,914), Revolution Brilliantpink-mini (U.S. Pat. No, Plant 6,899)) is decumbent type plant having long stems, a low plant height, abundant branching, and a high resistance to heat, cold and rain. However, there are only a few varieties having a great profusion of flowers, vivid purplish red color and small flower and a high resistance to rain, heat, cold and diseases. Accordingly, this invention was aimed at obtaining a new variety having a vivid purplish red color and small flowers, together with the above features.

The new variety of petunia plant according to this invention originated from a controlled crossing of a female parent; ‘CDS02’ (Petunia sellowiana Sendtu named ‘CD1’× a wild type of petunia plant native to Brazil named ‘A1’) and a pollen parent; ‘CDS01’ (Petunia sellowiana Sendtu named ‘CD2’× a wild type of petunia plant native to Brazil named ‘A2’). First, a series of controlled crossing was conducted with Petunia sellowiana Sendtu named ‘CD1’, and ‘CD2’, and a wild type of petunia plant native to Brazil named ‘A1’ and ‘A2’ in May 1993. From that crossing, 240 seedlings were obtained and 4 seedlings which including ‘CDS01’ and ‘CDS02’ were selected by March 1994. Then, 500 seedlings were obtained from a crossing of these 4 seedlings. 10 seedlings were selected by March 1995. These 10 seedlings were propagated by cutting, and grown as a trial, by flower bedding and in a container by October 1996. Finally, one seedling was selected and the botanical characteristics of that plant were examined. As a result, it was concluded that this new variety is distinguishable from other varieties, and then this variety of petunia plant was named ‘SUNBELKUOPI’.

The wild type petunia plants ‘CD1’, ‘CD2’, ‘CDS01’ and ‘CDS02’ are presently maintained at Oumi Nursery Center of SUNTORY Ltd. residing at 863-1, Aza-Iketani, Oomori-cho, Yokaichi-shi, Shiga-ken, Japan.

This new variety of petunia plant named ‘SUNBELKUOPI’ was asexually reproduced by cutting at the aforementioned Oumi Nursery Center of SUNTORY Ltd. and the homogeneity and stability thereof were confirmed.

In the following description, the color-coding is in accordance with The Horticultural Color Chart of The Royal Horticultural Society, London, England (R.H.S. Color Chart), and the Inter-Society color Council-Nation Bureau of Standards Color Name (I.S.C.C.-N.B.S. Color Name). A color chart based on The Japan Color Standard for Horticultural Plants (J.H.S. Color Chart) is also added for reference.

### SUMMARY OF THE VARIETY

Disclosed herein is a decumbent type petunia plant named ‘SUNBELKUOPI’ having very small vivid purplish pink flowers. The plant has abundant branching, a great profusion of blooms, and the whole bush remains in bloom for a considerable period of time. The plant is highly resistant to rain, heat, cold and disease. This new variety of petunia plant is suitable for potting, hanging pots and flower bedding.

### BRIEF DESCRIPTION OF THE DRAWINGS

FIG. 1 is photograph giving a partial view of the new variety of petunia plant named ‘SUNBELKUOPI’;

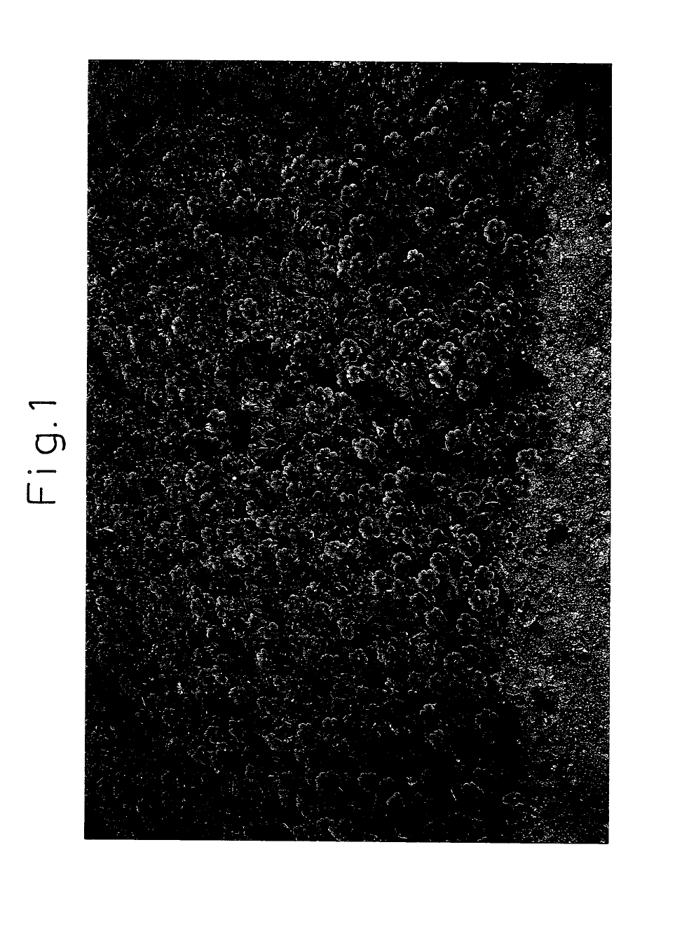

FIG. 2 is a photograph of flowers of the new variety of petunia plant named ‘SUBELKUOPI’

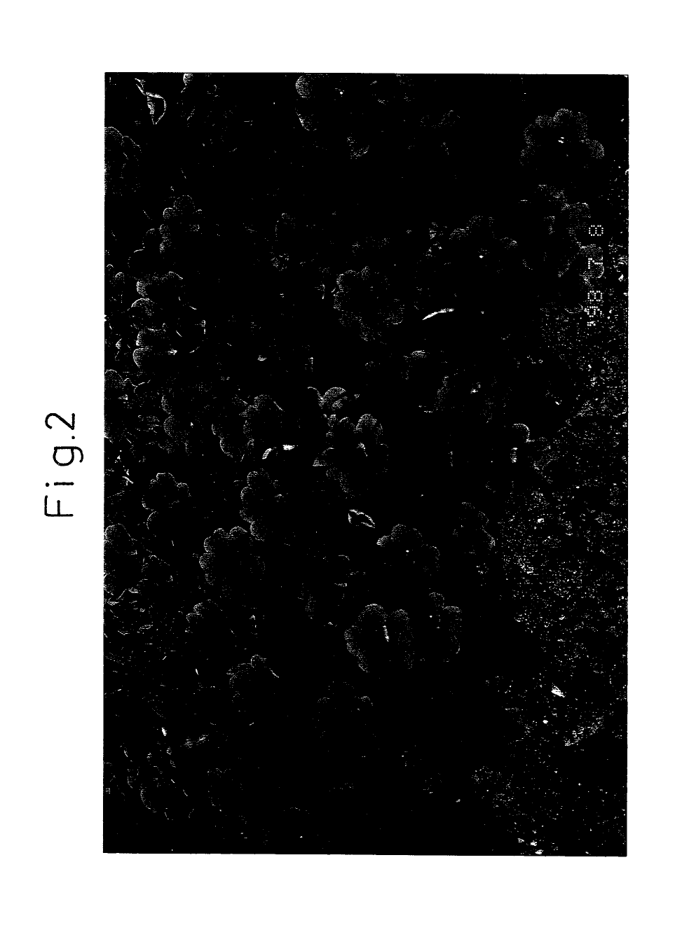

### DESCRIPTION OF THE VARIETY

The new variety of Petunia plant named ‘SUNBELKUOPI’ is different from a similar variety, ‘SUNBELKUPI’ in following points,

1. The flower color of ‘SUNBELKUOPI’ is vivid purplish pink (RHS C.C. No.80C) as against the color of ‘SANBELKUPI’ which is vivid reddish purple (RHS C.C. No.74A);

2. The shape of leaf of ‘SUNBELKUOPI’ is lanceolate to elliptic and wider than the leaf of ‘SANBELKUPI’;

3. The surface color of leaf of ‘SUNBELKUOPI’ is lighter than that of ‘SANBELKUPI’ and

4. The shape of petal tip of ‘SUNBELKUOPI’ is obverse as against the shape of petal tip of ‘SANBELKUPI’ which is medium.

The new variety of Petunia plant named ‘SUNBELKUOPI’ is different from a similar variety, ‘Pearl sky blue’ in following points;

1. The flower color of vivid purplish pink (RHS C. C.

No.80C) as against the color of ‘Pearl sky blue’ is bluish purple (RHS C.C. No.86C);

2. The shape of leaf of ‘SUNBELKUOPI’ is lanceolate to elliptic and wider than the leaf of ‘Pearl sky blue’,

3. The surface color of leaf of ‘SUNBELKUOPI’ is darker than that of ‘Pearl sky blue’ and

4. The flower size of ‘SUNBELKUOPI’ is smaller than that of ‘Pearl sky blue’. ‘SUNBELKUOPI’ is a decumbent plant, with small and vivid purplish pink flowers, which is suitable for potting, hanging pots and flower bedding. The spreading area of the plant is medium and the plant height is 5.0 cm at the blooming time of the 5^{th} to the 10^{th} flower. The diameter of the stem is 1.7 mm, with no anthocyanin coloration. This plant has abundant branching and secondary branching is especially strong. Length of internode is 1.5 cm. Leaves are very small, 3.1 cm in length, 1.2 cm in width, 0.4 mm in thickness. Leaf shape is lanceolate to elliptic, with some pubescence on the surface, having a small petiole. Leaf color is dark yellowish green (RHS C.C. No.146A). The flowers are single and very small, 2.8 cm in diameter. Facing direction of flower is slightly upward to horizontal. The color of flower is vivid purplish pink (RHS C.C. No.80C). The bottom color of inside of throat is bright greenish yellow (RHS C.C. No.2B) and the outside color of corolla tube is pale yellowish green (RHS C.C. No.150D). Shape of petal tip is obverse, lobation is shallow, and waving is weak. Shape of calyx is narrow. Shape of stamen is normal and number of stamens are 5. Shape of pistil is normal and number of pistils is one. Relative position of stigma and anthers is even. Length of peduncle is 1.9 cm and it is 0.8 mm in diameter.

Flowering time is late. The plant has high resistance to heat, rain, cold and diseases.

1. The new and distinct variety of Petunia plant, substantially as herein illustrated and described, characterized particularly as to novelty by (A) being a decumbent, (B) having an abundant branching and a great profusion of blooms, the whole bush remaining in bloom for considerable period of time, (C) having flowers that are single and very small, the petals having a vivid purplish pink color, and (D) having high resistance to rain, heat, disease.
